(19) United States
(12) Plant Patent Application Publication
(10) Pub. No.: US 2002/0002716 P1
(43) Pub. Date: Jan. 3, 2002

(54) APPLE TREE 'KITARO'

(75) Inventors: YOSHIO YOSHIDA, EBETSU-SHI (JP); SHICHIRO TSUCHIYA, RYUGASAKI-SHI (JP); JUNICHI SOEJIMA, MORIOKA-SHI (JP); TADAYUKI HANIUDA, NAGANO-SHI (JP); TETSURO SANADA, MORIOKA-SHI (JP); YOSHIKI KASHIMURA, TSUKUBA-SHI (JP); TETSUO MASUDA, MORIOKA-SHI (JP); HIDEO BESSHO, YAMANASHI-KEN (JP); SADAO KOMORI, ISHIGAKI-SHI (JP); YUJI ITO, IBARAKI-KEN (JP); KAZUYUKI ABE, MORIOKA-SHI (JP); NOBUHIRO KOTODA, MORIOKA-SHI (JP)

Correspondence Address: ARMSTRONG,WESTERMAN, HATTORI,, MCLELAND & NAUGHTON, LLP, 1725 K STREET, NW, SUITE 1000, WASHINGTON, DC, 20006, US

(73) Assignee: National Institute of Fruit Tree Science Ministry of Agriculture, Forestry and Fisheries, Ibaraki-ken (JP)

(21) Appl. No.: 09/270,841

(22) Filed: Mar. 18, 1999

(51) Int. Cl. A01H 5/00

(52) U.S. Cl. PLT/172

(57) ABSTRACT

Disclosed is an apple tree having a medium vigor, rounded and crenated leaves, exhibiting excellent resistance to Alternaria blotch and bearing a medium size fruit maturing mid-season and having high productivity with a slightly heavy preharvest fruit drop. The fruit has a oblate shape, has yellow colored skin and has excellent storage quality. The flesh of the fruit is yellowish white, juicy and firm, and further, has a strong sweetness and medium sourness, thereby providing a good sweet-sour balance and rich taste, and has an excellent eating quality.

### BACKGROUND OF THE VARIETY

The present invention relates to a new and distinctive variety of apple tree belonging to a medium-maturing apple cultivar and, more particularly, relates to an apple variety having high quality and high resistance to disease and insect, for eating raw and for processing.

Regarding medium-maturing apple varieties, although ‘Jonathan’ and ‘Starking Delicious’ were widely cultivated in the past, they have become unpopular due to their taste and poor storage quality, and there are no main medium-maturing apple varieties now. Thus it was necessary to breed a main medium-maturing apple variety immediately.

Accordingly, the present breeders crossed ‘Hatsuaki’, which is an excellent variety for both eating raw and for processing, with ‘Fuji’, which is a main economical variety, etc., to obtain excellent medium-maturing apple varieties having good fruit quality and high productivity. Namely, the purpose of the invention is to avoid russet on fruit surface and to improve the storage quality of the medium-maturing variety ‘Hatsuaki’ and to breed a new and distinct variety of medium-maturing apple having excellent disease and insect resistance for eating raw and for processing.

### Origin and Asexual Reproduction of the Variety

To accomplish this purpose, the breeders crossed ‘Fuji’, which had been cultivated at the Morioka branch of the Fruit Tree Research Station (currently the Apple Research Center, the National Institute of Fruit Tree Science), in Japan, and ‘Hatsuaki’ pollen in 1976 and obtained seeds. The seeds obtained were utilized for sowing and raising seedlings at said branch, in 1977, and the resulting seedlings were then grafted on ‘M.27’, the most dwarfing rootstock, to accelerate bearing in 1978, and 127 individuals were set in 1980.

The trees bore fruit in 1984 and one was selected in the primary selection as a good medium-maturing individual in 1986. The present invention is directed to an apple tree ‘Kitaro’, which is one individual from among the 127 individuals derived from the cross ‘Fuji’ (516 ) (the seed parent) and ‘Hatsuaki’ (♂) (the pollen parent), and having an individual number “512”. Then the tree was selected as a favorable line and the trees, which have a line name ‘Ringo Morioka 52’, have been provided for local adaptability tests since 1991, and were examined to determine the various characteristics thereof, over a period of eight years from April 1989 to March 1997. As a result, it was found that, for example, fruit of the trees had the desired characteristics, such as quality, storage quality and yield, as a medium-maturing apple variety, and the tree was distinguishable from the parent varieties, ‘Fuji’ and ‘Hatsuaki’, as well as a control variety ‘Golden Delicious’. Accordingly, the breeders denominated this variety as ‘Kitaro’, in accordance with this invention. The genus and species of the tree is ‘Malus×domestics’.

Of the varieties, ‘Fuji’ is a variety generated from a cross between ‘Rolls Janet’ and ‘Delicious’.

Of the varieties, ‘Hatsuaki’ is a variety generated from a cross between ‘Jonathan’ and ‘Golden Delicious.

The breeders asexually reproduced this new and distinctive variety of apple tree ‘Kitaro’, by grafting on rootstock ‘M.27’, at a number of Fruit Tree Research Stations, and confirmed the homogeneity and stability of ‘Kitaro’ according to the present invention.

An application for this new variety of apple tree, ‘Kitaro’ under the Seeds and Seedlings Law of Japan, was filed on Apr. 11, 1997, under the filing number 9766.

The original tree of this apple tree is held at the Apple Research Center of the National Institute of Fruit Tree Science, Ministry of Agriculture, Forestry and Fisheries, residing at 92 Nabeyashiki, Shimokuriyagawa, Morioka City, Japan.

### SUMMARY OF THE VARIETY

This new variety of apple tree, having medium vigor, is spreading, and many of spurs and axillary buds are formed.

The shape of a leaf of the tree is rounded and crenated.

At Morioka City, Iwate prefecture, Japan, the trees have a flowering time around May 21th. As the type of S gene of the present variety is (S3, S9), the cross-compatibility with common cultivars except ‘Hatsuaki’ and ‘Sekaiichi’ is high.

There is slightly heavy preharvest drop of fruit and the present variety exhibits high productivity. Regarding the main diseases, the tree is resistant to Alternaria blotch, but is susceptible to scab. It has not been recognized so far that any disease or insect etc. except for the above, is significant, if custom any controls are carried out.

The ripening of the present fruit occurs in mid October at Morioka, later by about one week than ‘Senshu’, and earlier by one week than ‘Golden Delicious’. The size of the fruit is medium and averages about 250-270 g, the same as ‘Senshu’. Although the type of ground color of the skin of the fruit is soft yellow-green and that of the over-color is light greenish yellow, the surface of the skin, receiving sunlight is colored with dull pink. The shape of the fruit is oblate and the amount of the russet on the skin is high, thus the appearance of the fruit is not good. Depending on the years, cracking occurs in the part of stalk cavity of the fruit.

The flesh of the present fruit contains much sugar, about 15-16% Brix of the total sugar, and about 0.5 g/100 ml of malic acid, and further the flesh is firm and juicy, has a strong sweetness and medium sourness and provides a good sweet-sour balance and rich taste. The storage quality of the fruit is excellent, and is about 15 days at room condition and more than 90 days in cold storage.

Compared with the seed parent ‘Fuji’, the present variety is distinguishable in that the ripening of the fruit is earlier by about 20 days than that of ‘Fuji’, the type of over color of the skin of the present variety is yellow, whereas that of ‘Fuji’ is colored with deep-reddish stripe, and the fruit of the present variety does not have tendency to an inclination of the axis, as does ‘Fuji’.

Compared with the pollen parent, ‘Hatsuaki’, the present variety is distinguishable in that flowering is earlier by several days and the ripening of the fruit is later by about 10-15 days than those of ‘Hatsuaki’, the color of the skin is yellow, whereas that of ‘Hatsuaki’ is reddish-orange, and the flesh of the present variety is firm, and the storage quality is excellent.

Compared with ‘Golden Delicious’, the present variety is clearly distinguishable in that the ripening of the fruit is earlier by about one week, and there is preharvest drop of the fruit, the shape of the fruit is flatter than that of ‘Golden Delicious’, the flesh is firm and the storage quality is excellent.

The color values presented are herein taken from the Japanese Horticultural Plant Standard Color Chart (JHSC).

### BRIEF DESCRIPTION OF THE DRAWINGS

FIG. 1 gives the pedigree of the new and distinct variety of the apple tree Kitaro;

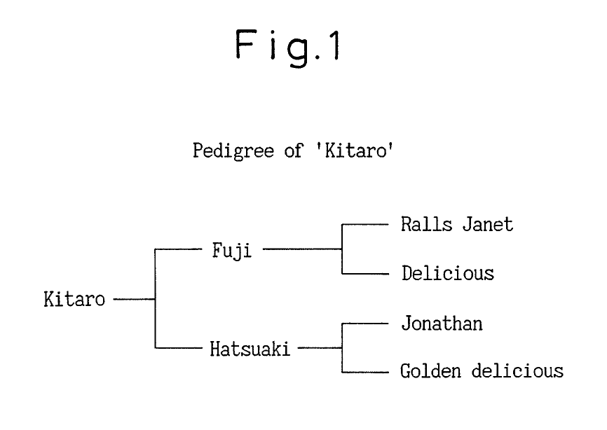

FIG. 2 is a photograph of a tree shape of the new variety of apple tree grafted on ‘M.9’ (8 years old);

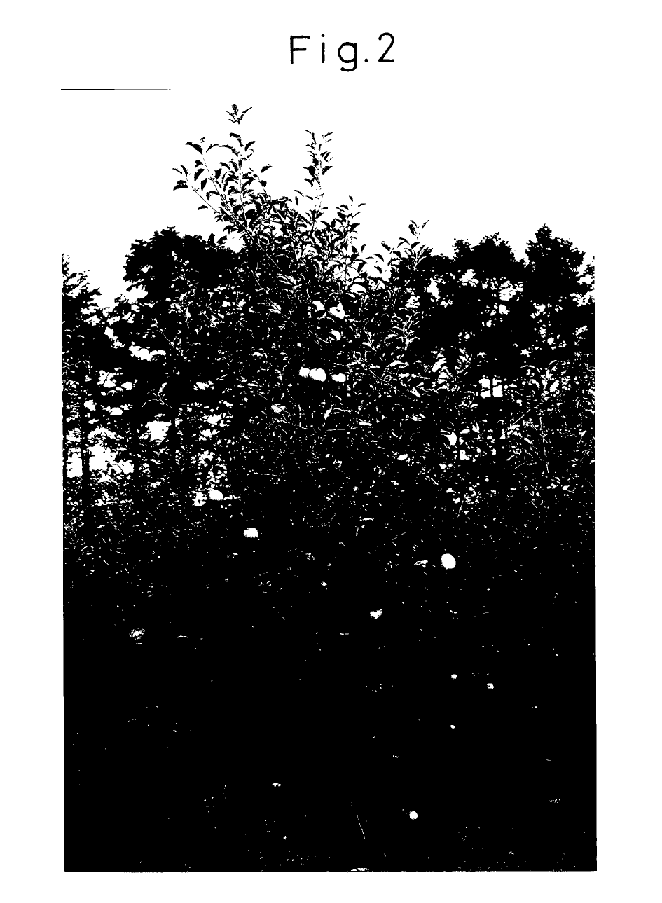

FIG. 3 is a photograph of adult leaves (upper and reverse side) of the new variety of apple tree;

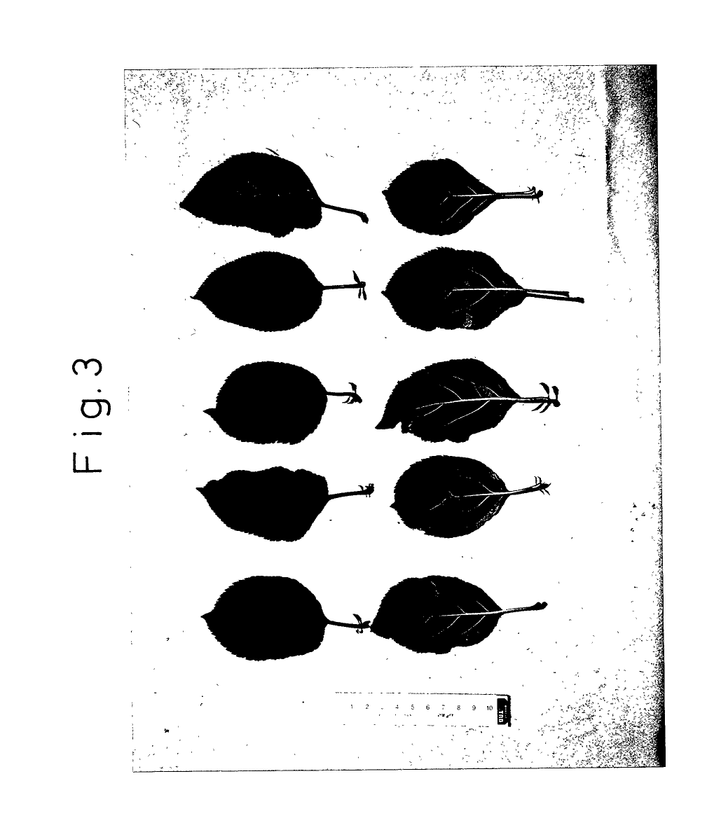

FIG. 4 is a photograph of the flowers (opened and unopened) of the new variety of apple tree;

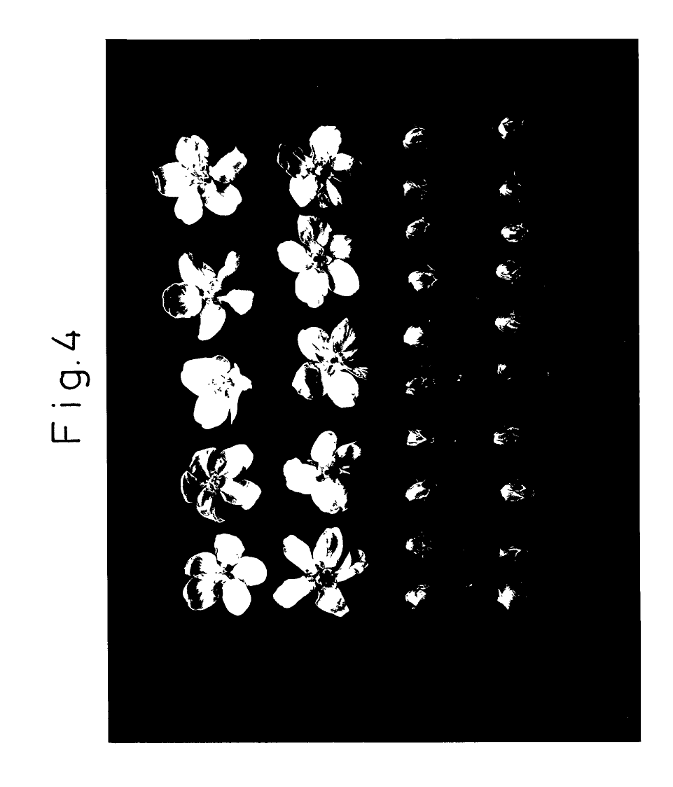

FIG. 5 is a photograph of the fruits on the tree of the new variety of apple tree;

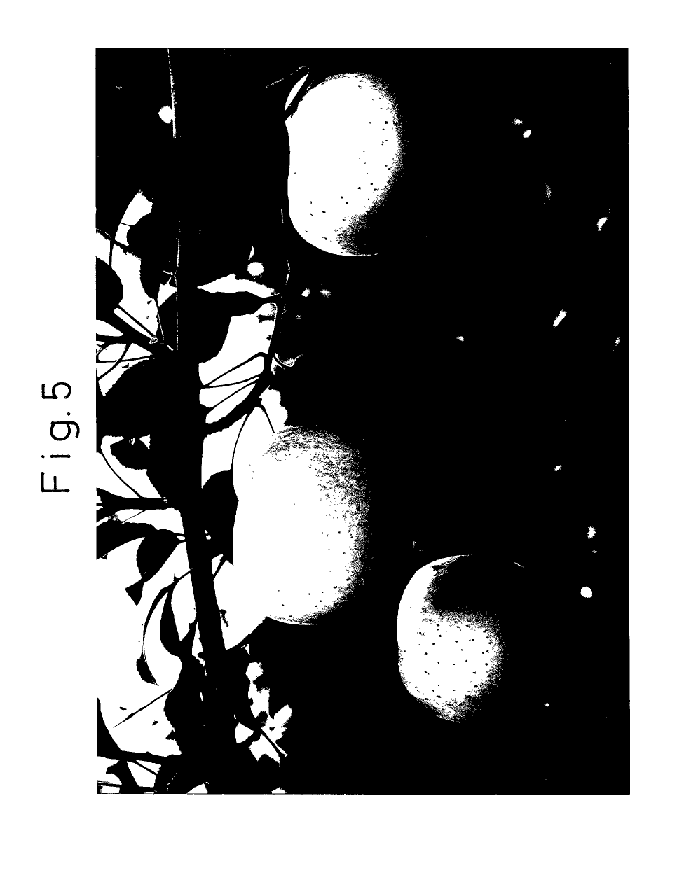

FIG. 6 is a photograph of side view, longitudinal-sectional view, and basin of the fruit of the new variety of apple tree;

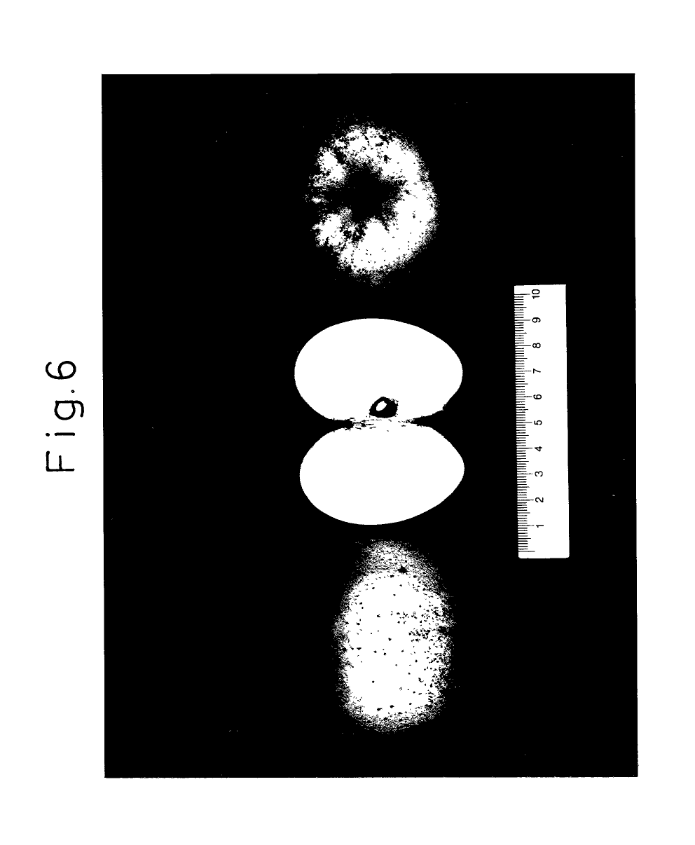

FIG. 7 is a photograph of longitudinal-sectional views of the fruit of the new variety of apple tree; and

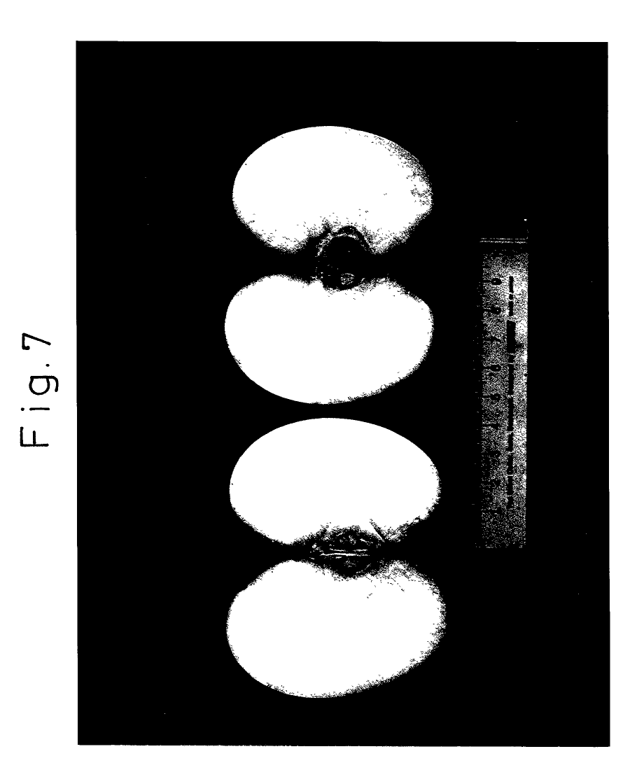

FIG. 8 is a photograph of cross-sectional views of the fruit of the new variety of apple tree.

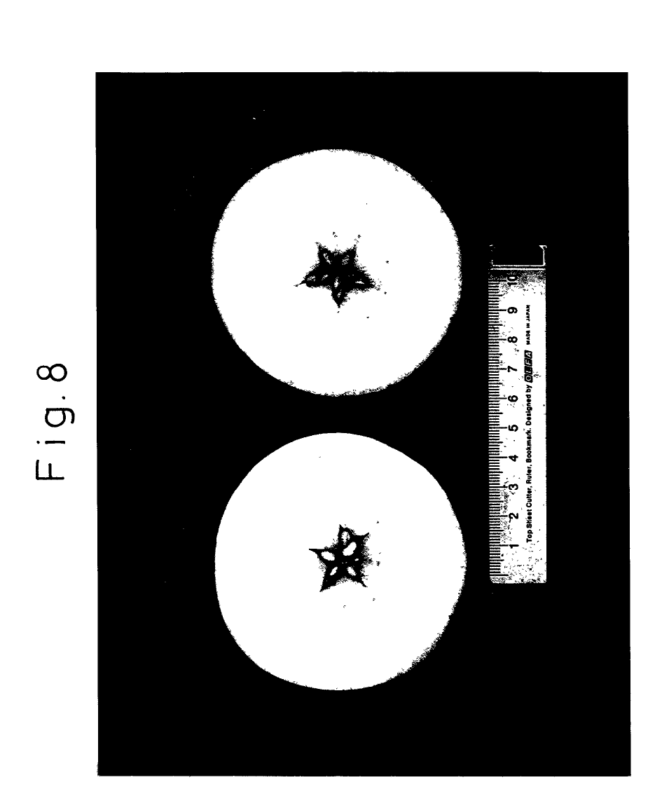

### DESCRIPTION OF THE VARIETY

The characteristics of the new and distinct variety of apple tree Kitaro are as follows:

Tree:

Habit of branches.—Medium and spreading, same as ‘Golden Delicious’.

Size.—Medium.

Vigor.—Medium.

Thickness (one-year-old shoot).—Medium.

Length of internode (on-year-old shoot).—Medium (average 2.8 cm).

Size of lenticels (one-year-old shoot).—Large.

Number of lenticels (one-year-old shoot).—Many (numerous).

Pubescence of shoot.—Weak.

Bearing habit of fruit.—On spur.

Bearing of axillary flower bud.—Many.

Leaf:

Shape of leaf blade.—Rounded, whereas that of ‘Golden Delicious’ is between Rounded and long.

Serrations of leaf edge.—Crenate, whereas that of ‘Golden Delicious’ is serrated.

Size of leaf (length).—Short (length average 8.4 cm, wide averadge 5.2 cm).

Color of leaf.—Dark Grayish-green.

Shape of stipules.—Sickle.

Size of stipules.—Short.

Length of petiols.—Medium.

Thickness of petiole.—Medium.

Flowers:

Number of flowers (per cluster).—Medium.

Size (open flower).—Medium.

Color (unopen flower).—Deep Pink.

Shape of petal.—Ovoid, whereas that of ‘Golden Delicious’ is oval.

Number of petals.—Medium (5).

Number of stamens.—Medium.

Color of anthers.—Light yellow.

Amount of pollen.—Much.

Fruit:

Shape.—Oblate, whereas that of ‘Golden Delicious’ is short-round conical.

Crowing at eye end.—Medium, that of ‘Golden Delicious’ is weak.

Aperture of eye.—Open.

Depth of basin (eye end).—Medium, that of ‘Golden Delicious’ is deep.

Breadth of basin (eye end).—Broad, that of ‘Golden Delicious’ is narrow.

Depth of stalk cavity.—Medium, same as ‘Golden Delicious’.

Breadth of stalk.—Broad, whereas that of ‘Golden Delicious’ is medium.

Size.—Small (about 250-270 g).

Type of ground color.—Soft yellow green, whereas that of ‘Golden Delicious’ is light yellow.

Type of over color of skin.—Light greenish yellow (JHSC 2904) with dull pink (JHSC 0412) on the cheek exposed to intense sunlight, whereas that of ‘Golden Delicious’ is light yellow.

Form of over color of skin.—Solid (undistinguished stripe).

Position of russet.—Overall.

Amount of russet.—Much.

Raised russet lenticels.—Present.

Size of lenticels.—Medium, whereas that of ‘Golden Delicious’ is large.

Number of lenticels.—Medium.

Silvery mottle of skin.—Absent.

Shininess of skin.—Medium, stronger than ‘Golden Delicious’.

Greasiness of skin.—Medium, more than ‘Golden Delicious’.

Bloom of skin.—Absent.

Cracking tendency of skin.—Present, whereas that of ‘Golden Delicious’ is absent.

Surface texture of skin.—Rough, rougher than ‘Golden Delicious’.

Length of stalk.—Long (about 3.0 cm).

Thickness of stalk.—Stout.

Distinct swelling at end of stalk.—None.

Shape of core.—Conical.

Size of core.—Small, (longitudinal section)

Number of core cells.—Medium (5 core cells) (loculus).

Color of flesh.—Yellowish white.

Bruising of flesh.—Difficult, whereas that of ‘Golden Delicious’ is easy.

Browning of flesh.—Strong, stronger than that of ‘Golden Delicious’.

Firmness of flesh.—Firm, firmer than that of ‘Golden Delicious’.

Texture of flesh.—Coarse, coarser than that of ‘Golden Delicious’.

Water core.—None or weak.

Sweetness of flesh.—Strong (Brix 15-16°), sweeter than ‘Golden Delicious’.

Acidity of flesh.—Medium (about 0.5 g/100 ml).

Astringency of flesh.—None.

Flavor of flesh.—Weak.

Juiciness of flesh.—Juicy.

Seed:

Number of fully developed seeds.—Medium (7.0).

Shape.—Ovovoid.

Size.—Large.

Physiological and ecological characteristics:

Date of germination.—Late (around April 10th in Morioka City, Iwate prefecture, Japan), later than that of ‘Golden Delicious’.

Season of flowering.—Late (around May 21th, in Morioka prefecture, Japan), later than that of ‘Golden Delicious’.

Time of fruit ripening for eating.—Medium (October 13 to 19th, in Morioka City, Iwate prefecture, Japan), earlier than that of ‘Golden Delicious’.

Time of fructification.—Precocious

Self-fruitfulness.—Weak (0%). (fructification by self-pollination).

Early dropping of fruit.—None or slight.

Preharvest dropping of fruit.—Heavy.

Physiological disorder of fruit.—Medium, cracking on part of the stalk cavity.

Storage quality of fruit (room condition).—Long (about 15 days).

Storage quality of fruit (cold storage).—Very long (about 90 days)

Occurrence of heart rot (core rot).—None or weak.

Resistance to Alternaria blotch.—Strong; stronger than ‘Golden Delicious’.

Culture: As the preharvest drop of the fruit of the new cultivar is slightly heavy, it is necessary to pay attention to optimum picking time and to avoid late picking. If the application of fruit drop regulators is approved, it is preferable to control fruit drop by spraying them.

The new variety ‘Kitaro’ will be widely planted, especially in northern part of Japan, as a late-maturing variety and a practical variety producing yellow fruit.

1. A new and distinct variety of apple tree, substantially as illustrated and described herein, characterized over known apple trees by (A) having a medium vigor and having leaves which are rounded and crenated, exhibiting excellent resistance to Alternaria blotch and (B) bearing a medium size fruit having a oblate shape, (a) medium-maturing, (b) having high productivity with a slight heavy preharvest fruit drop, and (c) having (1) excellent storage quality, (2) yellow colored skin, and (3) a yellowish white flesh that is juicy and firm has a strong sweetness and medium sourness, provides a good sweet-sour balance and rich taste and has an excellent eating quality.
